(19) United States
(12) Patent Application Publication
(10) Pub. No.: US 2025/0041531 A1
(43) Pub. Date: Feb. 6, 2025

(54) Assembly for an Injection Device and an Injection Device

(71) Applicant: Sanofi, PARIS (FR)

(72) Inventors: Max Davis, Melbourn, Hertfordshire (GB); Thomas Mark Kemp, Melbourn, Hertfordshire (GB); Tom Lever, Melbourn, Hertfordshire (GB); Max Wehebrink, Melbourn, Hertfordshire (GB); Robbie Wilson, Melbourn, Hertfordshire (GB)

(21) Appl. No.: 18/719,717

(22) PCT Filed: Dec. 13, 2022
(86) PCT No.: PCT/EP2022/085670

§ 371 Date: Jun. 13, 2024

(30) Foreign Application Priority Data
Dec. 15, 2021 (EP) 21315278.8

(51) Int. Cl. A61M 5/32; A61M 5/20

(52) U.S. Cl. CPC A61M 5/3245; A61M 5/3204; A61M 2005/208; A61M 2005/3267

(57) ABSTRACT

An assembly for an injection device, such as an auto-injector device, includes a housing comprising a first bearing surface to support a medicament container in a distal direction, and a needle cover comprising a second bearing surface to protrude from the housing further than a needle of the medicament container at least in a first state and in a first position. The needle cover is arranged in the housing to move telescopically in the housing when a force is exerted upon it in a proximal direction, and the needle cover comprises an opening for the needle to pass through when the needle cover is moved into the housing to a second position. The assembly also includes a coupling element to cause the medicament container to move into the proximal direction in parallel with the needle cover when the needle cover is moved into the proximal direction past the second position.

### CROSS REFERENCE TO RELATED APPLICATIONS

The present application is the national stage entry of International Patent Application No. PCT/EP2022/085670, filed Dec. 13, 2022, and claims priority to Application No. EP 21315278.8, filed Dec. 15, 2021, the disclosures of which are incorporated herein by reference.

### BACKGROUND

Various auto-injectors for the use in adults are known. Those comprise a needle that is configured to be inserted into a patient's body for a certain injection depth before releasing a medicament. Often, this certain injection depth is unfit for use in children and/or adults with certain conditions leading to thinner skin, e.g. a low BMI. Children have for example thinner skin than adults and the underlying tissue also is of lesser thickness when compared to adults. Therefore, a reduced injection depth is desirable.

### SUMMARY

The present disclosure relates to an improved assembly for an injection device, e.g. an auto-injector device, especially in regards to a reduced needle insertion depth.

In the present disclosure, an assembly for an injection device, e.g. an auto-injector device, is proposed. The assembly comprises a housing, the housing comprising a first bearing surface (or support bearing surface) configured to support a medicament container in a distal direction. The assembly further comprises a needle cover comprising a second bearing surface (or protruding bearing surface), e.g. on a distal end of the needle cover, configured to protrude from the housing further than a needle of the medicament container at least in a first state and in a first position, wherein the needle cover is arranged in the housing such as to move telescopically in the housing when a force is exerted upon it in a proximal direction. The needle cover comprises an opening for the needle to pass through when the needle cover is moved into the housing to a second position. The assembly also comprises a coupling element configured to cause the medicament container to move, e.g. to push the medicament container, into the proximal direction in parallel, e.g. in conjunction, in tandem and/or together, with the needle cover when the needle cover is moved into the proximal direction past the second position. During use of the injection device, the coupling element is configured to displace the medicament container axially from the first bearing surface into a proximal direction. Movement of the medicament container in the proximal direction may increase a distance of the medicament container to the first bearing surface in a proximal direction. During use of the injection device the coupling element may be configured to displace the medicament container axially from the first bearing surface into a proximal direction, e.g. thereby preventing insertion of the needle into the patient's body beyond a predetermined injection depth, e.g. an injection depth in a range between 2 mm and 7 mm, preferably in a range between 3 mm and 5 mm. The device may prevent intra muscular injection for kids or patients with thin skin.

The coupling element and the medicament container may be configured to move together in an axial direction, e.g. away from and/or towards the first bearing surface.

An injection device may be a device for injecting a medicament into the patient's body. Preferably, the injection device is a pen-type device. An auto-injector can be configured to provide a single shot of medicament, before being discarded. Preferably, the auto-injector is configured to completely or substantially empty the medicament container in the single shot. The auto-injector may comprise an energy storage, e.g. a drive spring, for expelling the medicament from the medicament container, wherein, for example, the energy of the energy storage may be released during a single activation/shot.

The assembly may comprise the medicament container. The medicament container may comprise a medicament container body and a needle protruding from a distal end of the medicament container body. The needle may be fluidly connected to or connectable to an interior of the medicament container body. The needle may be fixed to the medicament container body. The medicament container may be a syringe type medicament container. The medicament container body may comprise a tubular, e.g. cylindrical, form. The interior of the medicament container body may comprise a tubular, e.g. cylindrical, form. The medicament container may comprise a stopper configured to move in the interior of the medicament container to reduce a size of an enclosed compartment and to expel medicament from the interior of the medicament container, e.g. through the needle. The stopper may seal the interior of the container body proximally. The medicament container may be a syringe with a staked needle.

The first bearing surface may limit an axial motion of the medicament container in the distal direction. Proximal movement, e.g. in a limited fashion, may be allowed. The first bearing surface may be facing partially, mainly or entirely into a proximal direction. The first bearing surface may be provided directly on the housing or on an additional part that is fixed in regards to the housing at least in a distal direction or also in proximal direction. The medicament container may comprise a bearing surface, facing or mainly facing the first bearing surface and configured to contact the first bearing surface. The bearing surface of the medicament container may be arranged on the medicament container body, e.g. on a shoulder of the medicament container, e.g. in an area where the needle is fixed to the medicament container housing, alternatively on a radial flange of the medicament container body arranged at a proximal end of the medicament container body, e.g. opposite to the needle. Alternatively, the medicament container comprises an adapter part, housing the medicament container body and providing the bearing surface of the medicament container, e.g. in cases where a small diameter medicament container body is used

The needle cover may be configured to at least radially cover a part of the needle protruding from the housing at least in a first state. A force driving the needle cover into the housing in the proximal direction may be generated by pressing the second bearing surface against the patient's skin.

The coupling element may be rigid and/or non-deformable, e.g. when subjected to an axial force. Alternatively, the distancing element may at least partially be made of a compressible and/or soft material, e.g. to reduce impact forces upon the patient's skin. A compressibility of the distancing element may herein be chosen such that after fully inserting the needle up to the distancing element and activating a medicament discharge by a drive mechanism, an impact force of the drive mechanism may cause the distancing element to compress such that the needle is further inserted into the patient's body to reach the predetermined injection depth.

The proposed assembly allows the use of medicament containers with standardized needle length also for children.

According to further embodiments, the coupling element may be arranged on the first bearing surface and configured to be arranged between the first bearing surface and the medicament container, wherein the coupling element comprises a distal extension configured to extend along a needle of the medicament container. The needle cover may be covering a part of the distal extension protruding from the housing at least in a radial direction, at least in the first state.

Alternatively, the coupling element may be configured to be arranged on a needle of the medicament container or may be configured to be arranged on a neck of the medicament container body, in which the needle is affixed.

Furthermore, the coupling element may comprise a third bearing surface arranged on a distal end of the coupling element, e.g. on a distal end of the distal extension. The third bearing surface may comprise a surface area of at least 3 mm², e.g. at least 5 mm², e.g. at least 10 mm². E.g. the third bearing surface may have a ring shape with a diameter of 8.5 mm or more and a width of 0.5 mm or more, which may result in an area of 14 mm² or more. The third bearing surface and the opening may be dimensioned such that the third bearing surface can be arranged within the opening. The opening may be arranged in the second bearing surface. The third bearing surface may in the first position of the needle cover be arranged within the needle cover. The third bearing surface may be axially aligned with the second bearing surface at least in the second position of the needle cover.

Also, the third bearing surface may be configured to be inserted into the opening of the needle cover when the needle cover is moved into the second position and to contact the patient's skin when the needle cover reaches the second position. The opening may be configured to let the distal extension pass through when the needle cover is moved far enough into the housing, e.g. so that the distal extension may contact the patient's skin, e.g. in conjunction with the second bearing surface.

Alternatively, the third bearing surface may be configured to contact, e.g. abut, the needle cover, e.g. an inner bearing surface surrounding the opening of the needle cover, when the needle cover reaches the second position. In such a case the predefined injection depth may be defined by a difference of an offset from the needle tip and the third bearing surface and a thickness of the needle cover between the second bearing surface and the inner bearing surface of the needle cover.

According to further embodiments, the distal extension is formed as a hollow cylinder configured to surround at least a part of the needle. According to other embodiments, the distal extension has a cross-sectional form of a polygon or an oval.

Preferably, the third bearing surface is configured to be offset, e.g. completely and/or on average, from a needle tip of the needle axially, e.g. in a proximal direction, e.g. along the main axis, by the predefined injection depth. This allows for a smooth insertion of the needle into the patient's skin.

The assembly may further comprise a cap configured to cover a distal opening of the housing and comprising a grabber for removing a needle shield of the medicament container, wherein the grabber is configured to at least partially be arranged radially between the distal extension and the needle. The grabber may comprise a radially outer surface directed towards the distal extension. The grabber may comprise a radially inner surface directed towards the needle shield and/or the needle. This may allow for a smooth operation of the injection device.

Furthermore, the coupling element may comprise a proximal extension, configured to extend along the medicament container body of the medicament container. The proximal extension may be configured to surround the medicament container body form-fittingly in radial direction. The proximal extension may form a fitting sleeve for the medicament container body. This may allow for a secure holding of the medicament container.

According to further embodiments, the needle cover is configured to interact with a drive mechanism of the injection device to activate and/or enable a medicament ejection, e.g. when the needle cover arrives in the second position, e.g. in response to movement of the needle cover from the first position to the second position relative to the housing. Activation of the medicament ejection may lead to an immediate ejection of the medicament in response to the movement of the needle cover. Enablement of the medicament ejection may release a locking mechanism that is preventing an activation, while the actual activation may be triggered by a trigger element, e.g. an activation button, to be operated by the patient or a user separately from the needle cover. This may allow for a simple and/or safe operation of the injection device.

Preferably, the third bearing surface may be configured to contact the patient's skin, before the needle cover interacts with the drive mechanism to activate and/or enable the medicament ejection. This may allow for a simple and/or safe operation of the injection device. A transmission of an impact of the drive mechanism onto the patient's skin may be dampened by this.

The medicament container may be prefilled with a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N—(ω-carboxyheptadecanoyl)-des(B30) human insulin and B29-N—(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Furthermore, an injection device, e.g. an auto-injector device, comprising an earlier described assembly and a drive mechanism configured to eject medicament from the medicament container when activated, are proposed. The drive mechanism may comprise an energy storage. The energy storage may be pre-charged, e.g. during assembly of the injection device. The energy storage may be a mechanical energy storage, e.g. in form of a, e.g. compressed, spring. Alternatively the energy storage may be of electric or pneumatic nature, e.g. compressed gas. The auto-injector device may, in comparison to other injection devices, be configured to automatically perform an ejection of a medicament, e.g. by the drive mechanism, after a medicament discharge was initiated without requiring further actions of a user of the injection device. In alternative embodiments, a discharge of the medicament may include a constant acting of the user, e.g. an exertion of a driving force, even after initiation of the medicament discharge.

### BRIEF DESCRIPTION OF THE DRAWINGS

FIG. 1 shows a perspective and partially transparent view of an injection device comprising an assembly in a schematic view in a second state of operation.

FIG. 2a-d show cross-sectional views of the assembly in a first, the second, a third, and a fourth state of operation.

### DETAILED DESCRIPTION

In the figures, identical elements, identically acting elements or elements of the same kind may be provided with the same reference numerals.

“Distal” is used herein to specify directions, ends or surfaces which are arranged or are to be arranged to face or point towards a dispensing end of the drug delivery device or components thereof and/or point away from, are to be arranged to face away from or face away from the proximal end. “Proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

The distal direction may be the direction in which a tip of the needle is oriented to. A proximal direction may be a direction opposite the distal direction. The distal and proximal directions may be aligned along a main axis. The main axis may be colinear with the needle or run through the needle. Any or all moving components of the injection device may be configured to move along the main axis and/or rotate around the main axis. A radial direction may be any direction orthogonal to the main axis. The main axis may be an axis along which most of the components of the injection device are arranged and/or oriented. The main axis may be an axis of lowest moment of inertia of the injection device or at least close to such an axis.

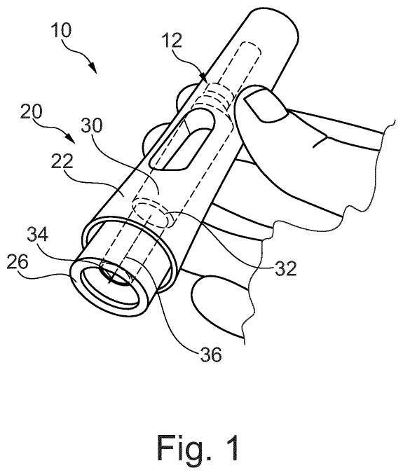

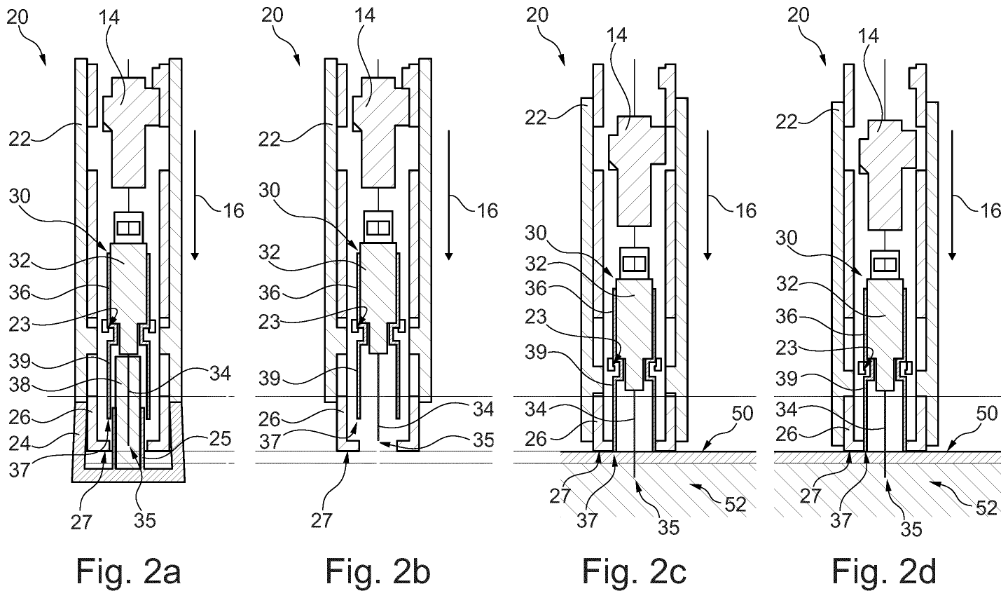

FIG. 1 shows an injection device 10. The injection device 10 is an auto-injector device. The injection device 10 is a pen-type device. The injection device comprises an assembly 20 (also compare FIGS. 2a-d) and a drive mechanism 12. The drive mechanism 12 may be part of the assembly. Details of the drive mechanism 12 may be found in the PCT patent application WO 2015/004052 A, the disclosure of which is herewith incorporated in this application. The drive mechanism may be the same or similar to the one disclosed in WO 2015/004052 A.

The assembly 20 for the injection device 10 is configured to comprise a medicament container 30. The assembly 20 includes the medicament container 30. The medicament container 30 comprises a medicament container body 32 and a needle 34 protruding from the medicament container body 32. The Needle 34 is fluidly connected to an interior of the medicament container body 32. The medicament container 30 is prefilled with a medicament. The drive mechanism 12 is configured to eject medicament from the medicament container 30 when activated. The drive mechanism 12 comprises an energy storage (not shown) and a plunger 14, wherein the energy storage is configured to drive the plunger towards and/or into the medicament container 30, e.g. in a distal direction 16. The plunger 14 is configured to interact with the medicament container 30 to eject medicament from the medicament container 30. The energy storage may be a compressed spring, e.g. arranged within the plunger 14.

The assembly 20 further comprises a housing 22. The housing comprises a first bearing surface 23 configured to support the medicament container 30 in the distal direction 16.

The assembly 20 further comprises a needle cover 26 comprising a second bearing surface 27 configured to protrude from the housing 22 further than a needle 34 of the medicament container 30 at least in a first state, wherein the needle cover 26 is arranged in the housing 22 such as to move telescopically in the housing 22 when a force is exerted upon it in a proximal direction, and wherein the needle cover 26 comprises an opening for the needle 34 to pass through when the needle cover 26 is moved into the housing 22 to a second position. The needle cover 26 is radially covering a part of the needle 34 protruding from the housing 22 at least in the first position of the needle cover 26, e.g. in the first and second state of operation (FIGS. 2a and 2b). The needle cover 26 comprises a cylindrical shape at least in the area where the needle 34 protrudes from the housing 22. The second bearing surface 27 is arranged on a distal end of the needle cover 26. The needle cover 26 may comprise a section that is formed on a distal end of the needle cover 26 and that extends radially inwards.

The needle cover 26 is arranged in the housing 22 such as to move telescopically in the housing 22 when a force is exerted upon it in the proximal direction, e.g. by pressing the second bearing surface 27 against the patient's skin 50. The needle cover 26 comprises a distal opening for the needle 34 and a coupling element 36 to pass through when the needle cover 26 is moved into the housing 22 to a second position or past the second position, e.g. in a third state of operation (compare FIG. 2c).

The assembly 20 further comprises the coupling element 36 configured to cause the medicament container 30 to move into the proximal direction in parallel with the needle cover 26 when the needle cover 26 is moved into the proximal direction past the second position. The coupling element 36 is configured to push the medicament container 30 into the proximal direction during use of the injection device 10, e.g. when the needle cover 26 is moved past the second position, such that the medicament container body 32 is proximally distanced to the first bearing surface 23.

The coupling element 36 is arranged on the first bearing surface. The coupling element 36 is configured to be arranged between the first bearing surface 23 and the medicament container body 32, e.g. along the distal direction 16. The coupling element 36 provides a third bearing surface 37 arranged on a distal end of the distal extension 39. The third bearing surface 37 is configured to be inserted into the opening of the needle cover 26 when the needle cover is moved into the second position and to contact a patient's skin 50 during use of the injection device 10, when the needle cover reaches the second position. Thereby, the coupling element 36 is preventing insertion of the needle 34 into the patient's body 52 beyond a predetermined injection depth.

The coupling element 36 comprises a distal extension 39 configured to extend along the needle 34 of the medicament container 30. The distal extension 39 extends from a bearing surface configured to provide support for the medicament container body 32 in the distal direction 16. The distal extension 39 extends parallel to the needle 34.

During use of the injection device 10 the coupling element 36 is configured to displace the medicament container 30 axially from the first bearing surface 23 into a proximal direction (opposite the distal direction 16).

The distal extension 39 is formed as a hollow cylinder configured to surround at least a part of the needle 34. The needle 34 is arranged along a center of the hollow cylinder. The needle 34 is arranged such that for any point along the needle, distances in any radial direction from the needle 34 to the distal extension 39 are constant.

The distal extension 39 is arranged along parts of the needle 34 that are to remain outside the patient' body 52 during use of the injection device.

The third bearing surface 37 is offset from a needle tip 35 of the needle 34 axially in the distal direction 16 by the predefined injection depth.

The coupling element 36 is made from an essentially rigid material. The coupling element 36, e.g. at least the distal extension 39, is made from a polymer and/or a metal, e.g. a medical grade polymer such as PC/ACBs, Nylon, Glass Filled Nylon, and/or POM, or a metal such as Stainless Steel would be appropriate. The third bearing surface may be formed as a flat surface, e.g. in comparison to an edge, wherein preferably the whole bearing surface is arranged in a plane. Alternatively conical shapes of the bearing surface are possible, e.g. to adapt to the patient's skin in a certain way.

The medicament container 30 further comprises a needle shield 38 configured to cover at least the needle tip 35, and a part of the needle 34. A portion of the needle shield 38 is arranged between the needle 34 and the coupling element 36, when viewed in a radial direction from the needle 34.

The needle shield 38 is configured to be removed before use of the injection device 10.

The assembly 20 further comprises a cap 24 to cover a distal opening of the housing 22. The cap 24 is releasably coupled to the housing 22. The cap 24 comprises a grabber 25 for removing the needle shield 38 of the medicament container 30. The grabber 25 is mechanically locked to the needle shield 38 such that the needle shield 38 is removed from the needle 34, when the cap 24 is removed from the housing 22. The grabber 25 is configured to partially be arranged radially between the distal extension 39 and the needle 34.

In a first state of operation (compare FIG. 2a) the cap 24 may still be attached to the housing 22. In a second state of operation the cap 24 and needle shield 38 are removed and the injection device 10 is ready for use (compare FIG. 2b).

The coupling element 36 further comprises a proximal extension, configured to extend along the medicament container body 32 of the medicament container 30. The proximal extension comprises a cylindrical form. The distal extension 39 comprises a first section and a second section. The first section and the second section may each comprise a hollow cylindrical form. The first section has a smaller diameter than the second section while having a same or similar wall thickness. The third bearing surface 37 is arranged on a distal end of the second section. The distal extension 39 may comprise a ring-shaped element arranged between a distal end of the first section and a proximal end of the second section to bridge a gap between the two sections. The coupling element 36 comprises a second ring shaped portion arranged between the distal extension 39, e.g. the first section, and the proximal extension. The first section comprises a smaller diameter than the proximal extension. The first section and the proximal extension comprise a same or similar wall thickness. The second ring shaped portion provides a distal bearing surface to interact with the first bearing surface 23 in the distal direction 16. The second ring shaped portion provides a proximal bearing surface to support the medicament container 30 in the distal direction 16.

The needle cover 26 is configured to interact with the drive mechanism 12 of the injection device 10 to activate and/or enable a medicament ejection. When the needle cover 26 reaches/arrives in a third position, the needle cover 26 loses engagement with the plunger 14.

In one embodiment, the energy storage may be configured to release stored energy to drive the plunger 14 into the distal direction to interact with the medicament container 30 to release the medicament as soon as the needle cover 26 loses engagement with the plunger 14. In another embodiment, after the needle cover 26 loses engagement with the plunger 14, an additional user action, e.g. a button press, may be implemented to release the stored energy and to move the plunger 14.

The third bearing surface 37 is configured to contact the patient's skin 50, before the needle cover 26 interacts with the drive mechanism 12 to activate and/or enable the medicament ejection.

In a fourth state of operation (FIG. 2d), a proximal bearing surface of the needle cover 26 may contact a distal bearing surface of the housing 22 such that a further movement of the needle cover 26 into the housing 22 is hindered. The fourth and third state of operation may be the same state of operation. In the fourth state of operation, the medicament container 30 is distanced proximally to the first bearing surface 23 further than in the third state of operation.

The needle cover 26 is covering a part of the distal extension 39 protruding from the housing 22 at least in the first state, e.g. also in the second state, wherein the opening is configured to let the distal extension 39 pass through when the needle cover 26 is moved far enough into the housing 22.

The assembly 20 and/or the injection device 10 may be provided by using a further assembly, wherein the further assembly comprises a medicament container 30, wherein the medicament container 30 comprises a medicament container body 32 and a needle 34 protruding from the medicament container body 32 and fluidly connected to an interior of the medicament container body 32, wherein the further assembly also comprises a housing 22 providing a first bearing surface 23 for supporting the medicament container 30 in a distal direction, wherein during use of the injection device 10, the needle 34 is configured to be inserted into a patient's body 52 up to a further predetermined injection depth, wherein a coupling element 36 is arranged between the medicament container 30 and the first bearing surface 23 that provides a third bearing surface 37 configured to contact the patient's skin 50, e.g. on a distal extension 39, during use of the injection device 10, thereby preventing insertion of the needle 34 into the patient's body 52 beyond a predetermined injection depth that is smaller than the further predetermined injection depth. The further assembly may comprise the housing 22, the needle cover 26, the cap 24 and/or the drive mechanism 12.

During production of the injection device, the medicament container 30 may be inserted into the coupling element 36, before or after the coupling element 36 is arranged in the housing 22.

### REFERENCE NUMERALS

10 injection device
12 drive mechanism
14 plunger
16 distal direction
20 assembly
22 housing
23 bearing surface
24 cap
25 grabber
26 needle cover
27 bearing surface
30 medicament container
32 medicament container body
34 needle
35 needle tip
36 coupling element
37 bearing surface
38 needle shield
39 distal extension

1-14. (canceled)

15. An assembly for an injection device, the assembly comprising:
a housing comprising a first bearing surface configured to support a medicament container in a distal direction,
a needle cover comprising a second bearing surface configured to protrude from the housing further than a needle of the medicament container in a first position of the needle cover, wherein the needle cover is arranged in the housing such as to move telescopically in the housing when a force is exerted upon it in a proximal direction, and wherein the needle cover comprises an opening for the needle to pass through when the needle cover is moved into the housing to a second position, and
a coupling element configured to cause the medicament container to move into the proximal direction in parallel with the needle cover when the needle cover is moved into the proximal direction past the second position.

16. The assembly according to claim 15, wherein the coupling element is arranged on the first bearing surface and configured to be arranged between the first bearing surface and the medicament container, wherein the coupling element comprises a distal extension configured to extend along the needle of the medicament container.

17. The assembly according to claim 16, wherein the distal extension is formed as a hollow cylinder configured to surround at least a part of the needle.

18. The assembly according to claim 17, comprising a cap configured to cover a distal opening of the housing and comprising a grabber for removing a needle shield of the medicament container, wherein the grabber is configured to at least partially be arranged radially between the distal extension and the needle.

19. The assembly according to claim 16, comprising a cap configured to cover a distal opening of the housing and comprising a grabber for removing a needle shield of the medicament container, wherein the grabber is configured to at least partially be arranged radially between the distal extension and the needle.

20. The assembly according to claim 15, wherein the coupling element comprises a third bearing surface arranged on a distal end of the coupling element.

21. The assembly according to claim 20, wherein the third bearing surface is configured to be inserted into the opening of the needle cover when the needle cover is moved into the second position and to contact skin of a patient when the needle cover reaches the second position.

22. The assembly according to claim 20, wherein the third bearing surface is configured to be offset from a needle tip of the needle axially by a predefined injection depth.

23. The assembly according to claim 20, wherein the third bearing surface is configured to contact skin of a patient, before the needle cover interacts with a drive mechanism to activate or enable medicament ejection.

24. The assembly according to claim 15, wherein the coupling element comprises a proximal extension, configured to extend along a medicament container body of the medicament container.

25. The assembly according to claim 15, wherein the needle cover is configured to interact with a drive mechanism of the injection device to activate or enable a medicament ejection.

26. The assembly according to claim 25, wherein a third bearing surface is arranged on the coupling element and is configured to contact skin of a patient, before the needle cover interacts with the drive mechanism to activate or enable the medicament ejection.

27. The assembly according to claim 15, further comprising the medicament container.

28. The assembly according to claim 27, wherein the medicament container is prefilled with a medicament.

29. The assembly according to claim 15, wherein the coupling element is at least one of rigid or non-deformable.

30. The assembly device according to claim 15, wherein the injection device is an auto-injector device.

31. An injection device comprising an assembly, the assembly comprising:
a housing comprising a first bearing surface configured to support a medicament container in a distal direction,
a needle cover comprising a second bearing surface configured to protrude from the housing further than a needle of the medicament container in a first position of the needle cover, wherein the needle cover is arranged in the housing such as to move telescopically in the housing when a force is exerted upon it in a proximal direction, and wherein the needle cover comprises an opening for the needle to pass through when the needle cover is moved into the housing to a second position, and
a coupling element configured to cause the medicament container to move into the proximal direction in parallel with the needle cover when the needle cover is moved into the proximal direction past the second position.

32. The injection device according to claim 31, wherein the injection device is at least one of a pen-type device or an auto-injector device.

33. The injection device according to claim 31, comprising a medicament container, and the medicament container is prefilled with a medicament.

34. A method of delivering a medicament from an injection device, the method comprising:
using the injection device, wherein the injection device comprises an assembly, and
wherein the assembly comprises:
a housing comprising a first bearing surface configured to support a medicament container in a distal direction,
a medicament container prefilled with a medicament,
a needle cover comprising a second bearing surface configured to protrude from the housing further than a needle of the medicament container at least in a first position of the needle cover, wherein the needle cover is arranged in the housing such as to move telescopically in the housing when a force is exerted upon it in a proximal direction, and wherein the needle cover comprises an opening for the needle to pass through when the needle cover is moved into the housing to a second position, and
a coupling element configured to cause the medicament container to move into the proximal direction in parallel with the needle cover when the needle cover is moved into the proximal direction past the second position.
